(19) United States
(12) Patent Application Publication
(10) Pub. No.: US 2014/0363493 A1
(43) Pub. Date: Dec. 11, 2014

(54) LIPID NANOPARTICLES FOR TARGETED siRNA DELIVERY

(71) Applicant: ALBERT EINSTEIN COLLEGE OF MEDICINE OF YESHIVA UNIVERSITY, Bronx, NY (US)

(72) Inventors: Deborah Palliser, Bronx, NY (US); Joseph Katakowski, Bronx, NY (US); Matthew Levy, Bronx, NY (US); Teresa DiLorenzo, Bayside, NY (US)

(73) Assignee: ALBERT EINSTEIN COLLEGE OF MEDICINE OF YESHIVA UNIVERSITY, Bronx, NY (US)

(21) Appl. No.: 14/299,194

(22) Filed: Jun. 9, 2014

Related U.S. Application Data
(60) Provisional application No. 61/833,072, filed on Jun. 10, 2013.

(51) Int. Cl. A61K 31/713; A61K 47/48

(52) U.S. Cl. CPC A61K 31/713; A61K 47/48823; A61K 47/48561
USPC 424/450; 424/135.1

(57) ABSTRACT

The present invention addresses a need for improved vehicles for delivering small interfering RNAs (siRNAs).

### CROSS-REFERENCE TO RELATED APPLICATIONS

This application claims benefit of U.S. Provisional Application No. 61/833,072, filed Jun. 10, 2013, the contents of which are hereby incorporated by reference.

### STATEMENT OF GOVERNMENT SUPPORT

This invention was made with government support under grant numbers R21 AI093539-01 and R01 AI099567-01 awarded by the National Institutes of Health. The government has certain rights in the invention.

### INCORPORATION BY REFERENCE

Throughout this application various publications are referred to in brackets. Full citations for these references may be found at the end of the specification. The disclosures of these publications, and of all patents, patent application publications and books referred to herein, are hereby incorporated by reference in their entirety into the subject application to more fully describe the art to which the subject invention pertains.

### BACKGROUND OF THE INVENTION

The discovery of a functional RNAi pathway in mammals has provided a powerful tool for reverse genetics as a method for identifying gene function. The potential of RNAi to silence any gene has also made it an attractive therapeutic modality [1]. However, the main obstacle to RNAi as a clinical agent is delivery. A delivery vehicle must transport its cargo through the body and, upon encountering cells must cross the plasma membrane and gain access to the cytosolic compartment, where the RNAi machinery resides. Furthermore, to be useful as a clinical reagent, ease of formulation and administration to the patient, overall cost and any associated toxicities must be considered.

Due to their ability to knockdown expression of any gene, siRNAs have been heralded as ideal candidates for treating a wide variety of diseases including “undruggable” targets. However, the therapeutic potential of siRNAs is severely limited by a lack of effective delivery vehicles. Recently, lipid nanoparticles (LNPs), containing ionizable cationic lipids, such as DLinDMA have been used to deliver siRNAs to the liver.

Currently, lipid nanoparticles (LNPs) are one of the most advanced delivery platforms being developed for systemic RNAi delivery [2]. LNPs contain ionizable cationic lipids that bind nucleic acids via electrostatic interactions, resulting in efficient siRNA encapsulation and a uniform LNP population of ˜100 nM diameter. Studies have also shown that following cellular uptake of the LNP, the ionizable lipid promotes siRNA escape from the endosome to the cytosol [3]. These properties make LNPs an attractive platform for siRNA delivery in vivo. LNPs are also associated with minimal toxicity, including little induction of pro-inflammatory cytokines following administration of physiologically relevant doses [4]. LNPs containing the ionizable lipid DLinDMA have been used for systemic delivery of siRNAs in mice, non-human primates (NHPs) and are in clinical trials [5]. Biodistribution studies show that systemic injection of LNPs results in accumulation mainly in the liver and spleen, and these LNPs are being evaluated in the clinic for conditions that require hepatic gene silencing [5,6]. The ED₅₀ (the dose required to observe 50% gene silencing) in liver-expressed genes following DLinDMA-formulated LNPs is ˜1 mg/kg³. Modifications to DLinDMA have resulted in identification of more potent lipids. Injection of murine clotting factor VII-specific siRNAs encapsulated in LNPs formulated with the lipid 1,2-dilinoleyl-4-(2-dimethylaminoethyl)[1,3]-dioxolane (DLinKC2-DMA), resulted in gene-specific silencing with an ED₅₀ of 0.1 mg/kg³. Additional modifications to this lipid identified the dilinoleyl-methyl-4-dimethylaminobutyrate (DLin-MC3-DMA) with a potency of 0.01 mg/kg when incorporated into siRNA-containing LNPs [2].

These LNPs provide a well-characterized, potent, minimally toxic system for siRNA delivery to cells in the liver and spleen. Splenic delivery makes these LNPs attractive candidates for silencing in immune cell types that are well represented in the spleen such as T cells, B cells, macrophages (MOs) and dendritic cells (DCs), thereby opening up the possibility of using LNPs for delivery of immune-modulatory siRNAs. Recently, Cullis and colleagues used a range of LNPs, including those containing DLinDMA and DLin-KC2-DMA, to determine efficacy of RNAi-mediated gene silencing in MOs and DCs in vitro and in vivo. Systemic injection of DLin-KC2-DMA-containing LNPs encapsulating an siRNA specific for GAPDH resulted in uptake by MOs and DCs isolated from the peritoneum and spleen. LNP uptake was shown to induce RNAi-mediated gene-specific silencing [7]. This study, together with a complementary study [8], indicates that gene silencing in immune cells using the LNP technology is feasible. However, the main target for systemically administered LNPs is the liver. Using such a non-targeted approach suffers from potential drawbacks including the possibility of toxicity if genes are silenced in the liver as well as in the desired splenic immune cell population(s). Furthermore, these studies showed a requirement for injection of relatively high doses of siRNA to achieve effective gene silencing in splenic DC and/or MO populations (3-5 mg/kg [7]).

The present invention provides a facile method of adapting LNPs for specifically targeted siRNA delivery.

### SUMMARY OF THE INVENTION

The present invention provides improved vehicles and methods for delivering siRNAs or other small cargoes.

This invention provides a composition comprising a lipid nanoparticle which comprises a lipid bi-layer, and a lipid having a single-chain variable fragment (scFv) attached thereto via a hydrophilic polymer wherein the scFv is directed against an antigen present on the surface of a cell.

Also provided is a method of delivering a small molecule, a nucleic acid, an antigen or a vaccine to a cell expressing a cell-surface antigen, comprising contacting the cell with any of the compositions described herein, wherein the cell surface antigen is the antigen to which the scFv is directed, in an amount effective to deliver a small molecule, a nucleic acid, an antigen or a vaccine to a cell expressing a cell-surface antigen.

Also provided is a method of increasing the efficacy of a predetermined dose of a nucleic acid, such as an RNAi nucleic acid, administered to a subject comprising delivering the nucleic acid to a cell expressing a cell-surface antigen, comprising contacting the cell with any of the compositions described herein, wherein the cell surface antigen is the antigen to which the scFv is directed, in an amount effective to deliver the nucleic acid to a cell expressing a cell-surface antigen and thereby increase the efficacy of the predetermined dose of a nucleic acid.

Also provided is a method of treating an immune system disorder in a subject comprising administering to the subject an amount of any of the compositions described herein, wherein the siRNA is an immunomodulatory siRNA or the small organic molecule is an immunomodulatory small organic molecule. In embodiment, the subject is a human.

### BRIEF DESCRIPTION OF THE DRAWINGS

FIG. 1: A diagram showing components of a targeted DLinDMA liposome as disclosed herein.

FIG. 2: A diagram showing a liposome designed to target specifically the DEC205 receptor.

FIGS. 3A-3B: Liposome quality metrics. FIG. 3A shows diameters of liposomes. FIG. 3B shows rates of siRNA incorporation into liposomes. FIG. 3C shows conjugation of liposomes to scFv.

FIG. 4: Graphs showing uptake of liposomes by DCs in vitro.

FIG. 5: Graphs showing uptake of liposomes by DCs in vivo.

FIG. 6: Specificity of delivery of scFv-liposomes in vivo.

FIG. 7: Uptake of NT-liposomes in vivo.

FIG. 8: Uptake of scFv-liposomes in vivo.

FIG. 9: Gene-specific silencing with scFv-liposomes in vivo.

FIG. 10: 2′-OMe modification of siRNAs diminishes dendritic cell activation following delivery via scFv DEC205-LNPs.scFv DEC205-LNPs encapsulating siRNAs (luciferase-specific) that were either unmodified (black), 2′-F (medium gray) or 2′-OMe (dark gray) were injected intravenously into mice. LNP uptake was tracked by presence of rhodamine B (DOPE-rhodamine B sulfonyl). 24 hrs later CD11c+ CD8α+ splenic DCs were isolated and analyzed for LNP uptake (top panel: uptake) and expression of costimulatory molecules CD40, CD86 and CD80 by flow cytometry. Expression levels are shown as mean fluorescence intensity. Data was analyzed by unpaired student's t-test. Error bars show s.e.m., ***P<0.001 and ****P<0.0001. Groups of 5 mice were used, apart from the untreated group which was one mouse only ( ). 2′-F=2′-floro group; 2′-OMe=2′-O Methyl group.

FIG. 11A-C: DEC205-targeted LNPs are specifically taken up by DEC205+ cells and co-localize to lysosomes. A. BMDC from WT and DEC205−/− were incubated with LNPs coated with either scFv DEC205 (green), scFv isotype (black), no scFv (blue) or nothing (red). Cells were incubated at 4° C. (left panel) or 37° C. (right panel) for 30 mins, washed and analyzed by flow cytometry. B. DEC205−/− and WT mice were injected with scFv-DEC205 LNPs or nothing. LNP-injected mice: Black=homozygous knockout, Blue=heterozygous knockout, green=WT; untreated mice: red=WT. Top panel shows DEC205 expression, bottom panel LNP uptake (gated on CD11c+CD8α+ cells). C. scFv DEC205− and scFv isotype control-coated LNPs containing fluorescently-labeled siRNAs were incubated for 1 hr, 37° C. with A20 cells. Following fixation and permeabilization cells were stained with EEA-1 and LAMP-1 and cellular localization of LNPs determined by confocal microscopy. DEC205-LNP, Iso-LNP=green; EEA-1, LAMP-1=red. Images shown acquired with 63× objective.

FIG. 12A-D: In situ uptake of scFv-coated LNPs results in gene-specific knockdown that is mediated via the RNAi pathway. C57BL/6 mice were injected with 0.7 mg/kg scFv DEC205-coated LNPs, containing either CD80 or control siRNAs. After 24 hrs spleens were harvested. LNPs contained Cy3-labeled siRNA to monitor cell uptake. A. LNP uptake by CD11c+DEC205+ cells (left panel) and CD80 expression in CD11c+DEC205+ DCs (right panel) was determined by flow cytometry. Red=no treatment, green=CD80 siRNA, blue=control siRNA. B. and C. CD11c+CD8α+Cy3+ cells were isolated by FACS. Total RNA was extracted and CD80 mRNA levels were determined by qPCR (b) and 5′ RACE analysis was performed to confirm RNAi-specific mRNA cleavage (c). PCR products were resolved by gel electrophoresis. Lane 1=siRNA CD80; Lane 2=siRNA control; Lane 3=100 bp ladder. Arrow shows ˜190 bp product. D. C57BL/6 mice (3/group) were injected with siRNAs specific for CD86 (CD86) or a non-target control (CN) followed by LPS, 6 hrs later. One group was injected with LPS alone (LPS) or was left untreated (UN). One day later, splenocytes were stained for CD11c, CD8α, CD80 and CD86. The left panel shows CD86 expression on CD11c+CD8α+ cells derived from mice that were untreated (red), CD86 siRNA (green), control siRNA (blue) or LPS only (orange). Numbers within graph are MFI values, each line represents one mouse. Data from all mice are quantified in the bar graphs. Significance was determined by one-way ANOVA. *, P<0.05; **, P<0.01; ns=not significant.

FIG. 13: Immune stimulation by siRNAs is dependent on siRNA modification. C57BL/6 mice (3 mice/group) were injected with DEC205-coated LNPs containing either siRNA that was unmodified (UN), 2′-F-(F) or 2′-OMe-(ME) modified or were not injected (NT). One day later spleens were isolated and expression of CD80, CD86 and CD40 determined on CD11c+CD8α+ cells. Statistical significance was determined by one-way ANOVA. **, P<0.01; ***, P<0.001; ****, P<0.0001; ns=not significant.

FIG. 14A-B: Immune stimulation by siRNAs is dependent on siRNA modification and experimental assay. A. BMDCs derived from C57L/6 mice were cultured with non-target LNPs containing 2′F-modified control siRNA (black) or CD80 siRNA (light blue), DEC-coated LNPs containing 2′F-modified control siRNA (dark blue) or CD80 (green), LPS (orange) or nothing (red) for 24 hrs. Expression of CD80, CD86 and CD40 was determined by flow cytometry. B. Cytokines produced by human PBMCs following incubation with LNPs containing siRNAs with various modifications and gene specificities. Table summarizes results of a dose-response assay testing PBMCs from three healthy donors. Most cytokines were detected only at the highest concentration of siRNA tested (5 mM).

FIG. 15. Injection of DEC205-LNPs containing siRNAs targeting costimulatory molecules reduces DEC205+ DC-mediated MLR. C57L/6 mice were injected with DEC205-LNPs containing either a mixture of siRNAs targeting CD80, CD86 and CD40 (Mix) or non-targeted siRNA (Ctrl). To enhance the MLR response, four hours following DEC205-LNP injection all groups of mice, except an untreated group, were injected with LPS. The following day DEC205+ DCs were isolated and co-cultured with T cells derived from a Balb/c mouse. Two days later T cell activation was determined by CFSE dilution. Graph shows the relative stimulation index, which compares the level of proliferation observed in LPS-treated mice (set to 1) with the other treatment groups. Untx=untreated, LPS=LPS only, Ctrl=control siRNA in DEC205-LNP, Mix=CD80, CD86 and CD40 siRNA in DEC205-LNP. Statistical significance was calculated by t-test. **, P<0.01; ***, P<0.001.

### DETAILED DESCRIPTION OF THE INVENTION

The present invention provides improved vehicles and methods for delivering siRNAs or other small cargoes.

This invention provides a composition comprising a lipid nanoparticle which comprises a lipid bi-layer, and a lipid having a single-chain variable fragment (scFv) attached thereto via a hydrophilic polymer wherein the scFv is directed against an antigen present on the surface of a cell.

Single-chain variable fragments are known in the art as a fusion protein of the variable regions of the heavy (VH) and light chains (VL) of immunoglobulins, connected with a short linker peptide of ten to about 25 amino acids. The linker is usually rich in glycine for flexibility, as well as serine or threonine for solubility, and can either connect the N-terminus of the VH with the C-terminus of the VL, or vice versa. In an embodiment, the scFv is monovalent. In an embodiment, the scFv is bivalent, directed against two different cell-surface antigens (e.g. two of CD80, CD86 and CD40).

In an embodiment, the composition comprises an ionizable cationic lipid. In an embodiment, the composition comprises distearoylphosphatidylcholine (DSPC). In an embodiment, the composition comprises the ionizable cationic lipid comprises 1,2-DiLinoleyloxy-N,N-dimethylaminopropane (DLinDMA).

In an embodiment, the composition comprises one or more of 1,2-dilinoleyl-4-(2-dimethylaminoethyl)-[1,3] -dioxolane (DLinKC2-DMA), dilinoleyl-methyl-4-dimethylaminobutyrate (DLin-MC3-DMA), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl phosphatidyl ethanolamine, 16-O-dimethyl phosphatidyl ethanolamine, 18-1-trans phosphatidyl ethanolamine, 1-stearoyl-2-oleoyl-phosphatidyethanolamine (SOPE), N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), and N,N-dimethyl-2,3-dioleyloxy)propylamine (DODMA), and 1,2-Dilinolenyloxy-N,N-dimethylaminopropane (DLendMA).

In an embodiment, the lipid having the hydrophilic polymer attached comprises distearoyl-phosphatidyl-ethanolamine-polyethylene glycol (DSPE-PEG). In an embodiment, the lipid nanoparticle comprises at least one interior lumen. In an embodiment, the lipid bi-layer encapsulates at least one interior lumen.

In an embodiment, (i) a nucleic acid molecule, (ii) a small organic molecule, (iii) a vaccine or (iv) an antigen is present in the lipid nanoparticle. In an embodiment, (i) a nucleic acid molecule, (ii) a small organic molecule, (iii) a vaccine or (iv) an antigen is present in an interior lumen of the lipid nanoparticle.

In an embodiment, the nucleic acid molecule is present and is a siRNA, shRNA, plasmid, mRNA, miRNA or ncRNA. In embodiment, the siRNA or shRNA is not 2′-fluoro modified.

In embodiment, the siRNA or shRNA is 2′-OMe modified.

In an embodiment, the scFv is an anti-DEC-205 scFv, anti-Cd11c scFv, anti-33d1 scFv or anti-Clec9A (DNGR1) scFv In an embodiment, the lipid nanoparticle comprises DSPC, DLinDMA, DSPE-PEG-malemide and cholesterol.

In an embodiment, the composition comprises 15% DSPC, 40% DLinDMA, 5% DSPE-PEG-malemide and 40% cholesterol.

In an embodiment, the interior lumen is aqueous.

In an embodiment, the hydrophilic polymer of the lipid having the hydrophilic polymer attached is PEG, and the scFv is attached to the PEGylated lipid via a covalent bond to a maleimide bonded to the PEG. In an embodiment, the bond is a —C—S— bond. In an embodiment, the scFv comprises a C-terminal cysteine, a Vl and a Vh, and the C-terminal cysteine is attached to the Vl or the Vh of the scFv. In an embodiment, the bond is between the maleimide attached to the PEG and a C-terminal cysteine of the scFv.

In an embodiment, the scFv is attached to the hydrophilic polymer of the lipid via a azide/alkyne covalent bond or an amide bond.

In an embodiment, the cell is a mammalian cell. In an embodiment, the mammalian cell is human. In an embodiment, the cell is a dendritic cell.

Also provided is a method of delivering a small molecule, a nucleic acid, an antigen or a vaccine to a cell expressing a cell-surface antigen, comprising contacting the cell with any of the compositions described herein, wherein the cell surface antigen is the antigen to which the scFv is directed, in an amount effective to deliver a small molecule, a nucleic acid, an antigen or a vaccine to a cell expressing a cell-surface antigen.

A method of increasing the efficacy of a predetermined dose of a nucleic acid administered to a subject, such as an RNAi nucleic acid, comprising delivering the nucleic acid to a cell expressing a cell-surface antigen, comprising contacting the cell with any of the compositions described herein, wherein the cell surface antigen is the antigen to which the scFv is directed, in an amount effective to deliver a nucleic acid to a cell expressing a cell-surface antigen and thereby increase the efficacy of the predetermined dose of a nucleic acid. In an embodiment, the nucleic acid is a siRNA or shRNA.

Also provided is a method of treating an immune system disorder in a subject comprising administering to the subject an amount of any of the compositions described herein, wherein the siRNA is an immunomodulatory siRNA or the small organic molecule is an immunomodulatory small organic molecule. In embodiment, the subject is a human.

In an embodiment, the immune system disorder is an autoimmune disease. Autoimmune diseases include acute disseminated encephalomyelitis (ADEM), alopecia areata, antiphospholipid syndrome, autoimmune cardiomyopathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticarial, autoimmune uveitis, Behcet's disease, celiac disease, Chagas disease, cold agglutinin disease, Crohn's disease, dermatomyositis, diabetes mellitus type 1, eosinophilic fasciitis, gastrointestinal pemphigoid, Goodpasture's syndrome, Grave's syndrome, Guillain-Barré syndrome, Hashimoto's encephalopathy, Hashimoto's thyroiditis, lupus erythematosus, Miller-Fisher syndrome, mixed connective tissue disease, myasthenia gravis, pemphigus vulgaris, pernicious anaemia, polymyositis, psoriasis, psoriatic arthritis, relapsing polychondritis, rheumatoid arthritis, rheumatic fever, Sjogren's syndrome, temporal arteritis, transverse myelitis, ulcerative colitis, undifferentiated connective tissue disease, vasculitis, and Wegener's granulomatosis.

In an embodiment of the methods, the cell is a dendritic cell. In an embodiment of the methods, the nucleic acid is delivered.

In embodiment of the methods or compositions, the nucleic acid is an RNAi nucleic acid. In an embodiment of the methods, the nucleic acid is a siRNA, shRNA, plasmid, mRNA, miRNA or ncRNA. In an embodiment, the siRNA or shRNA is not 2′-fluoro modified. In an embodiment, the siRNA or shRNA is 2′-fluoro modified. In embodiment, the siRNA or shRNA is 2′-OMe modified. In embodiment, the siRNA or shRNA is not 2′-OMe modified. In embodiment, the nucleic acid is an immune-modulatory siRNA. In embodiment of the methods or compositions, the RNAi nucleic acid is directed against a nucleic acid encoding CD80 (for example, encoding NCBI Reference Sequence: NP_{—}005182.1), CD40 (for example, encoding NCBI Reference Sequence: NP_{—}001241.1), CD86 (for example, encoding NCBI Reference Sequence: NP_{—}001193853.1), MyD88, TRIF, TRAF6, IRAK, RIG-1, MDA-5, p38, ERK, jnk, PDL1, or IRAK-M. In a preferred embodiment, said RNAi nucleic acid is an siRNA. In embodiment, the nucleic acid is a gene. In embodiment, said gene is a human gene.

In an embodiment, the siRNA or shRNA is directed against a mammalian CD80. In an embodiment, the siRNA or shRNA is directed against a mammalian CD86. In an embodiment, the siRNA or shRNA is directed against a mammalian CD40.

In an embodiment, the siRNA (small interfering RNA) as used in the methods or compositions described herein comprises a portion which is complementary to an mRNA sequence encoded by NCBI Reference Sequence for the stated genes/proteins. In an embodiment, the siRNA comprises a double-stranded portion (duplex). In an embodiment, the siRNA is 20-25 nucleotides in length. In an embodiment the siRNA comprises a 19-21 core RNA duplex with a one or 2 nucleotide 3′ overhang on, independently, either one or both strands. In an embodiment, the overhang is UU. The siRNA can be 5′ phosphorylated or not and may be modified with any of the known modifications in the art to improve efficacy and/or resistance to nuclease degradation. In a non-limiting embodiment, the siRNA can be administered such that it is transfected into one or more cells.

In one embodiment, a siRNA of the invention comprises a double-stranded RNA comprising a first and second strand, wherein one strand of the RNA is 80, 85, 90, 95 or 100% complementary to a portion of an RNA transcript of a gene (e.g. encoding human CD80, CD40, or CD86). Thus, in an embodiment, the invention encompasses an siRNA comprising a 19, 20 or 21 nucleotide first RNA strand which is 80, 85, 90, 95 or 100% complementary to a 19, 20 or 21 nucleotide portion, respectively, of an RNA transcript of a gene (e.g. encoding human CD80, CD40, or CD86). In embodiment, the second RNA strand of the double-stranded RNA is also 19, 20 or 21 nucleotides, respectively, a 100% complementary to the first strand. In another embodiment, a siRNA of the invention comprises a double-stranded RNA wherein one strand of the RNA comprises a portion having a sequence the same as a portion of 18-25 consecutive nucleotides of an RNA transcript of a gene (e.g. encoding human CD80, CD40, or CD86). In yet another embodiment, a siRNA of the invention comprises a double-stranded RNA wherein both strands of RNA are connected by a non-nucleotide linker. Alternately, a siRNA of the invention comprises a double-stranded RNA wherein both strands of RNA are connected by a nucleotide linker, such as a loop or stem loop structure.

In one embodiment, a single strand component of a siRNA of the invention is from 14 to 50 nucleotides in length. In another embodiment, a single strand component of a siRNA of the invention is 14, 15, 16, 17, 18, 19, 20, 21, 22, 23, 24, 25, 26, 27, or 28 nucleotides in length. In yet another embodiment, a single strand component of a siRNA of the invention is 21 nucleotides in length. In yet another embodiment, a single strand component of a siRNA of the invention is 22 nucleotides in length. In yet another embodiment, a single strand component of a siRNA of the invention is 23 nucleotides in length. In one embodiment, a siRNA of the invention is from 28 to 56 nucleotides in length.

In an embodiment, an siRNA of the invention comprises at least one 2′-sugar modification. In an embodiment, an siRNA of the invention comprises at least one 2′-O-fluorine modification. In an embodiment, an siRNA of the invention comprises at least one 2′-O-methyl modification. In an embodiment, an siRNA of the invention comprises at least one nucleic acid base modification. In an embodiment, an siRNA of the invention comprises at least one phosphate backbone modification.

In one embodiment, RNAi inhibition of the relevant gene is effected by a short hairpin RNA (“shRNA”). The shRNA is introduced into the cell by transduction with a vector inside the liposome. In an embodiment, the vector is a lentiviral vector. In an embodiment, the vector comprises a promoter. In an embodiment, the promoter is a U6 or H1 promoter. In an embodiment the shRNA encoded by the vector is a first nucleotide sequence ranging from 19-29 nucleotides complementary to the target gene, in the present case relevant gene. In an embodiment the shRNA encoded by the vector also comprises a short spacer of 4-15 nucleotides (a loop, which does not hybridize) and a 19-29 nucleotide sequence that is a reverse complement of the first nucleotide sequence. In an embodiment the siRNA resulting from intracellular processing of the shRNA has overhangs of 1 or 2 nucleotides. In an embodiment the siRNA resulting from intracellular processing of the shRNA overhangs has two 3′ overhangs. In an embodiment the overhangs are UU. The shRNA may comprise one or more 2′-O-fluorine modification and/or one or more 2′-O-methyl modification.

In an embodiment, the delivery of the small molecule or nucleic acid is selective to a cell expressing the cell-surface antigen for which the scFv is directed. In an embodiment, the delivery of the small molecule or nucleic acid is selective to a cell expressing the cell-surface antigen for which the scFv is directed if the small molecule or RNAi molecule is delivered to a cell expressing the cell-surface antigen at a rate 5-fold or greater than it is to a cell not expressing the cell-surface antigen under otherwise the same conditions. In an embodiment, the delivery of the small molecule or nucleic acid is selective to a cell expressing the cell-surface antigen for which the scFv is directed if the small molecule or RNAi molecule is delivered to a cell expressing the cell-surface antigen at a rate 10-fold or greater than it is to a cell not expressing the cell-surface antigen under otherwise the same conditions. In an embodiment, the delivery of the small molecule or nucleic acid is selective to a cell expressing the cell-surface antigen for which the scFv is directed if the small molecule or RNAi molecule is delivered to a cell expressing the cell-surface antigen at a rate 20-fold or greater than it is to a cell not expressing the cell-surface antigen under otherwise the same conditions. In an embodiment, the delivery of the small molecule or nucleic acid is selective to a cell expressing the cell-surface antigen for which the scFv is directed if the small molecule or RNAi molecule is delivered to a cell expressing the cell-surface antigen at a rate 35-fold or greater than it is to a cell not expressing the cell-surface antigen under otherwise the same conditions.

In an embodiment, the lipid nanoparticles of the invention comprise:
DSPC 11-20%
DLinDMA 35-45%
DSPE-PEG-Mal 2.5-7.5%
Cholesterol 25-35%.

In a preferred embodiment t, the lipid nanoparticles of the invention comprise:
DSPC 15%
DLinDMA 40%
DSPE-PEG-Mal 5%
Cholesterol 40%.

Preferred PEG sizes are PEG MW 2000. In an embodiment the PEG is MW 500-5000. Alternatively, PEG can be replaced in the compositions and methods described herein, mutatis mutandis, by other hydrophilic polymers.

Malemide linkages as described in the compositions herein can, alternatively, be substituted with amides, Click chemistry such as azide/alkyne, Staudinger chemistry and reactive animation.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Results

Injection of siRNAs encapsulated in DLinDMA-based LNPs, decorated with a single chain antibody (scFv) specific for DEC-205 targeting moiety, resulted in delivery of the siRNA cargo specifically to DEC-205-positive cells. Uptake of siRNA led to gene-specific knockdown via the RNAi pathway. The results suggest that targeting the siRNA to a specific cell subset can reduce the effective dose by at least one log when compared with non-targeted counterparts. Furthermore, restricting siRNA uptake to distinct cell subsets provides a means of limiting undesirable effects in bystander cells.

To achieve DC-directed gene silencing LNPs were modified by attaching a single chain antibody (scFv) via the PEG linker. The scFv specific for the DEC205 receptor was chosen for various reasons. First, DEC205 is expressed at high levels on DCs [9]. Second, uptake of an antigen coupled to anti-DEC205 antibody mediates effective cross-presentation, suggestive that cargoes taken up through the DEC205 pathway gain access to the cytosolic compartment [10]. As much of the RNA-induced silencing complex (RISC) resides in the cytosol this is an important consideration for choosing an appropriate receptor for shuttling siRNAs into a cell.

Systemic injection of scFv DEC205-decorated LNPs resulted in uptake that was limited to DEC205+ cells. Furthermore, delivery of siRNAs specific for CD80, a costimulatory molecule expressed by activated DCs, resulted in a reduction of expression by at least 50%.

The observed gene knockdown is likely mediated via the RNAi pathway, and this can be confirmed using 5′ RACE (to detect appropriately sized mRNA cleavage products). Anti-CD80 transfection of siRNA resulted in protein knockdown via RNAi in vitro.

This system can be extended to include additional gene targets of interest. Here, DC costimulatory molecules are targeted as a method to treat autoimmune diseases. This study has also identified siRNAs that effectively reduce CD86 and CD40 expression in vitro (data not shown). These sequences can be delivered by the LNPs disclosed herein in vivo.

Targeting DCs is significant because of the immune-modulatory capacity of this cell type. Knocking down inhibitory genes in DCs is useful for potentiating immune responses e.g. for use as a prophylactic or therapeutic vaccine. Conversely, silencing stimulatory genes is useful for treatment of autoimmune diseases.

### Results

### EXAMPLE 1

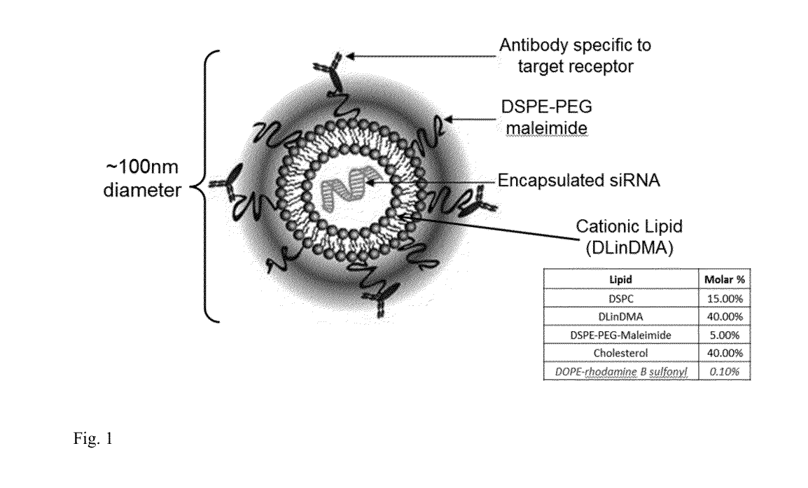

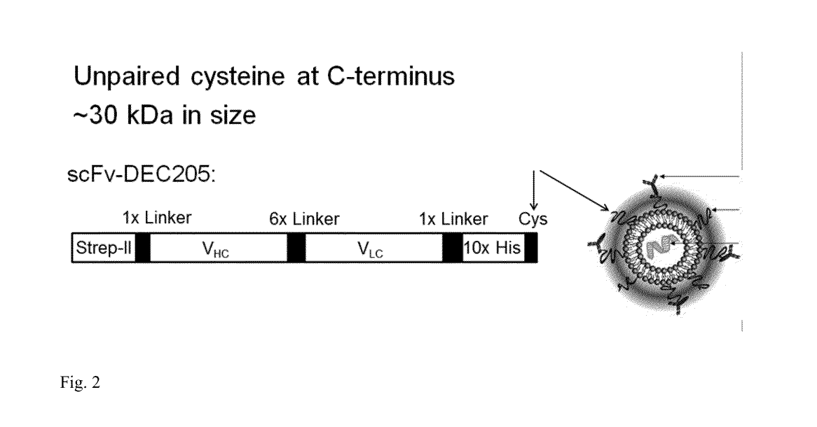

LNPs containing siRNAs specific either for CD80 or a control (non-target) sequence were synthesized. The formulation of the LNPs are DSPC:DLinDMA:DSPE-PEG:cholesterol 15:40:5:40. Anti-DEC205 (murine) scFv with a cysteine at its C-terminal end was attached to the lipid, DSPE-PEG by a maleimide group. A schematic is shown in FIGS. 1 and 2.

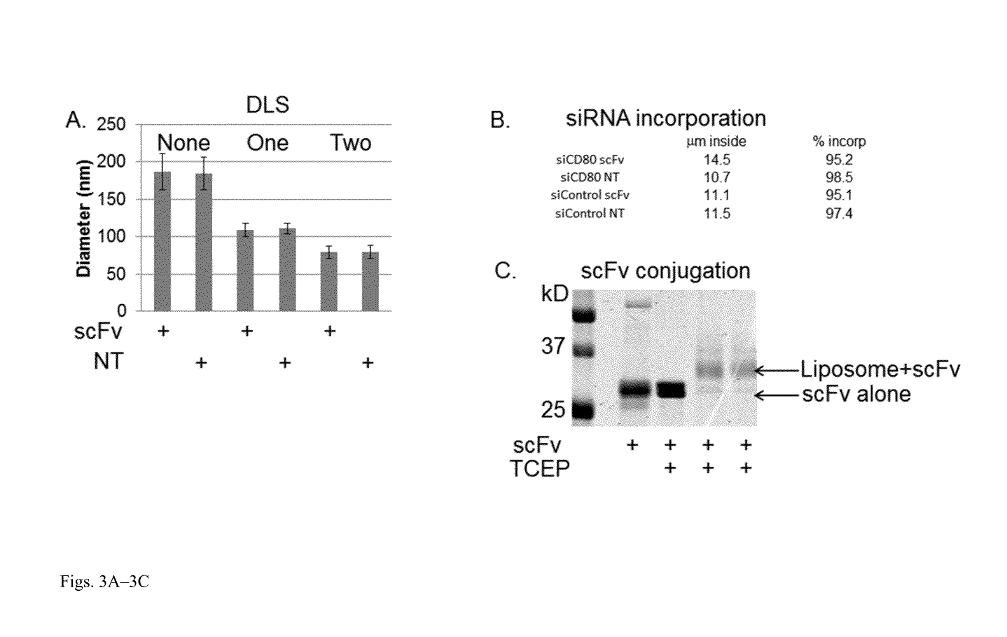

Following synthesis, LNPs are subject to quality control, to ensure consistency between batches. LNP diameter (dynamic light scattering) and the level of siRNA incorporation and scFv binding to DSPE-PEG were measured. Analysis of a typical set of LNPs is shown in FIG. 3.

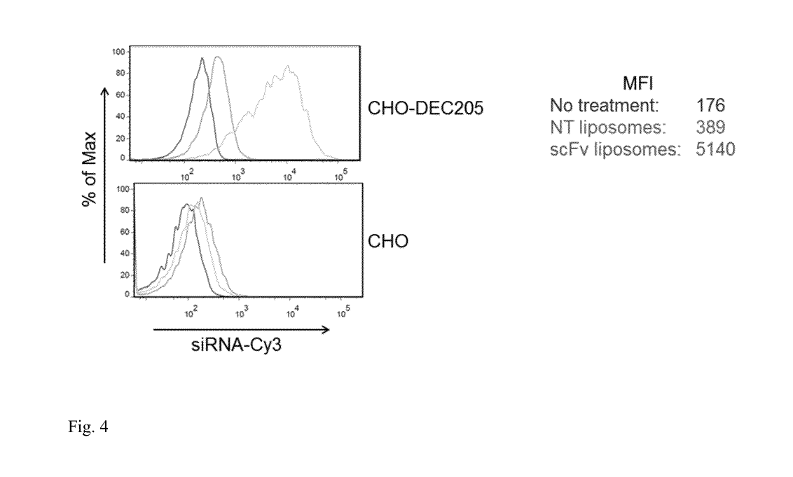

To show that scFv-LNPs selectively bound to DEC205, scFv-LNPs that encapsulated Cy3-labeled siRNA were cultured with CHO cells that stably expressed DEC205 or with parental CHO cells. As seen in FIG. 4, analysis by flow cytometry shows that cells that expressed DEC205 took up scFv-LNPs >10-fold better compared with LNPs that were not coated with DEC205-specific scFv (Top panel. Mean Fluorescence Intensity (MFI): non-targeted LNPs=389 versus DEC205 scFv-LNPs=5140). Little uptake was observed when LNPs (targeted or non-targeted) were incubated with parental CHO cells.

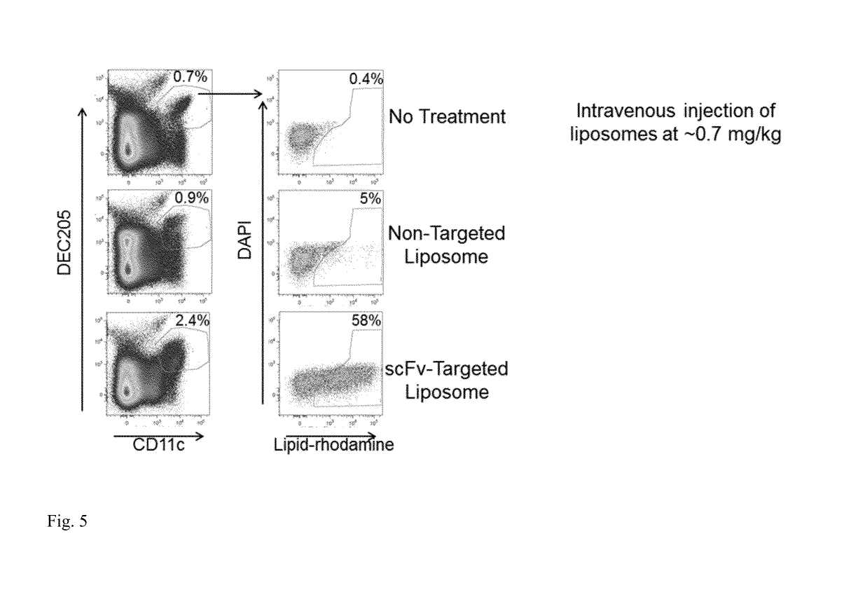

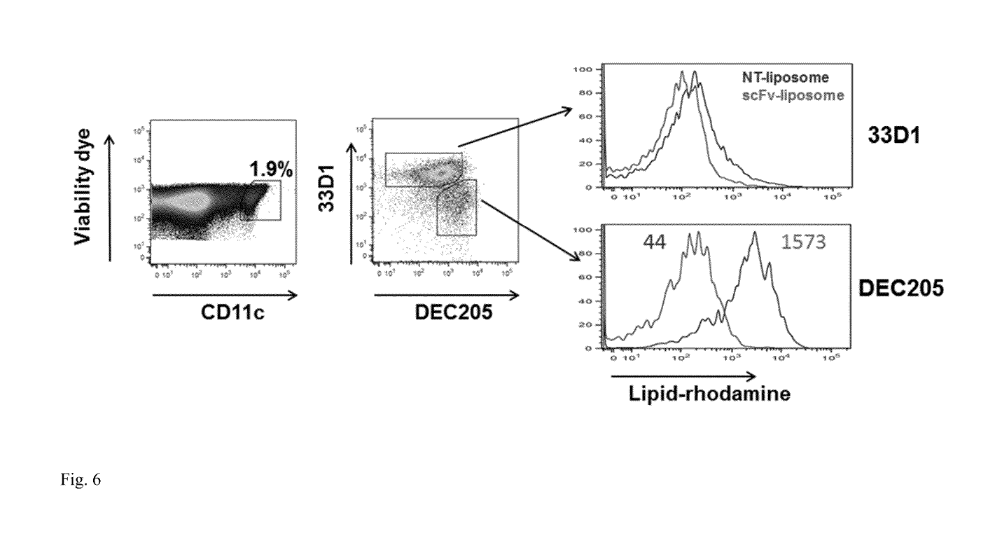

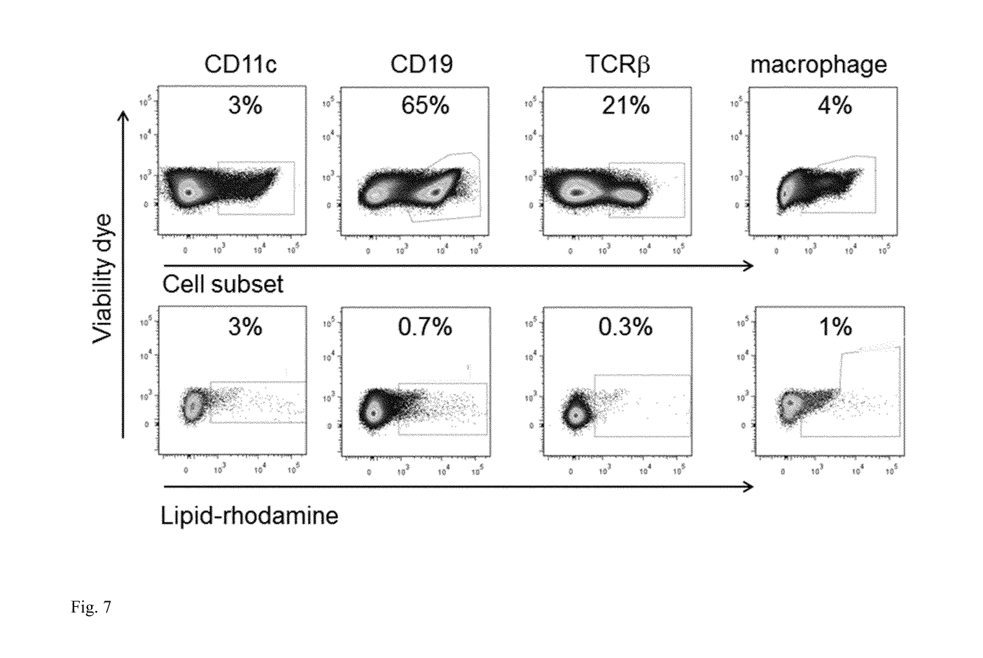

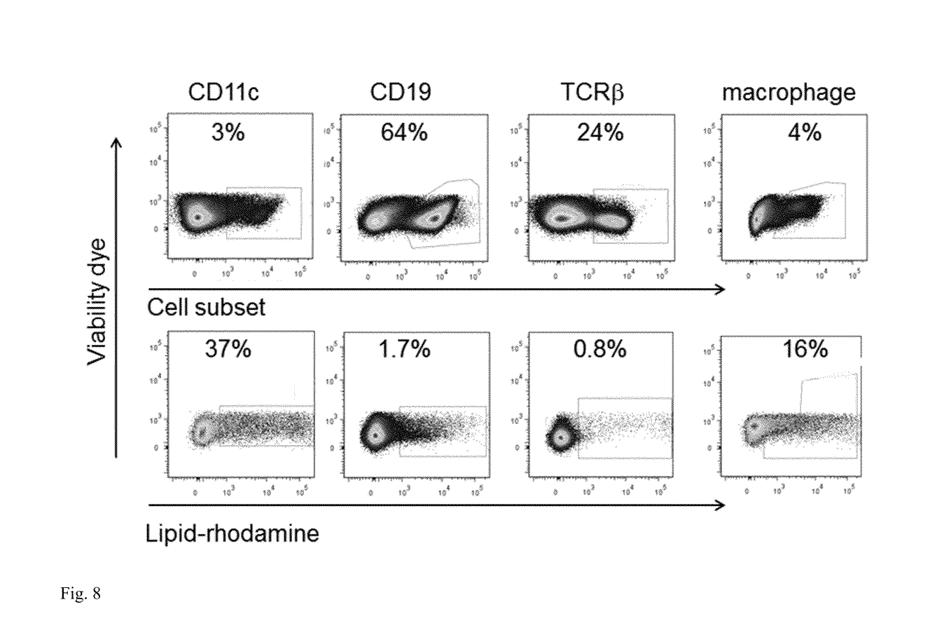

Having shown that the scFv-LNPs are specifically taken up by DEC205+ cells in vitro (including by DCs, data not shown), one goal was to determine whether this observation extended in vivo. C57L6 mice were injected systemically (intravenous, i.v.), with ˜0.7 mg/kg scFv-LNPs. These LNPs were synthesized with the inclusion of DOPE-rhodamine B sulfonyl (0.1%) to track lipid uptake. After 24 hrs, spleens were removed and uptake and distribution of LNPs was determined by flow cytometry. As seen in FIG. 5, ˜60% of CD11c+ DEC205+ cells are rhodamine-positive, indicative of scFv-LNPs (bottom panel, right side). DCs are not one homogenous population and in the spleen they can be further divided into CD11c+ DEC205+ and CD11c+ 33D1+ cells. As seen in FIG. 6, uptake of LNPs coated with DEC205 scFv is restricted to DEC205+ DCs (bottom panel, right side), with little uptake observed in the 33D1+ DCs (top panel, right side). Further analysis was undertaken to demonstrate specificity of uptake to DEC205-expressing cells. As seen in FIG. 7, injection of non-targeted LNPs at a low dose (0.7 mg/kg) resulted in little uptake by any splenic immune cell (including DCs). In contrast, injection of scFv-LNPs resulted in uptake by splenic DCs (as shown in FIG. 5), with little uptake by other splenic immune cells (FIG. 8).

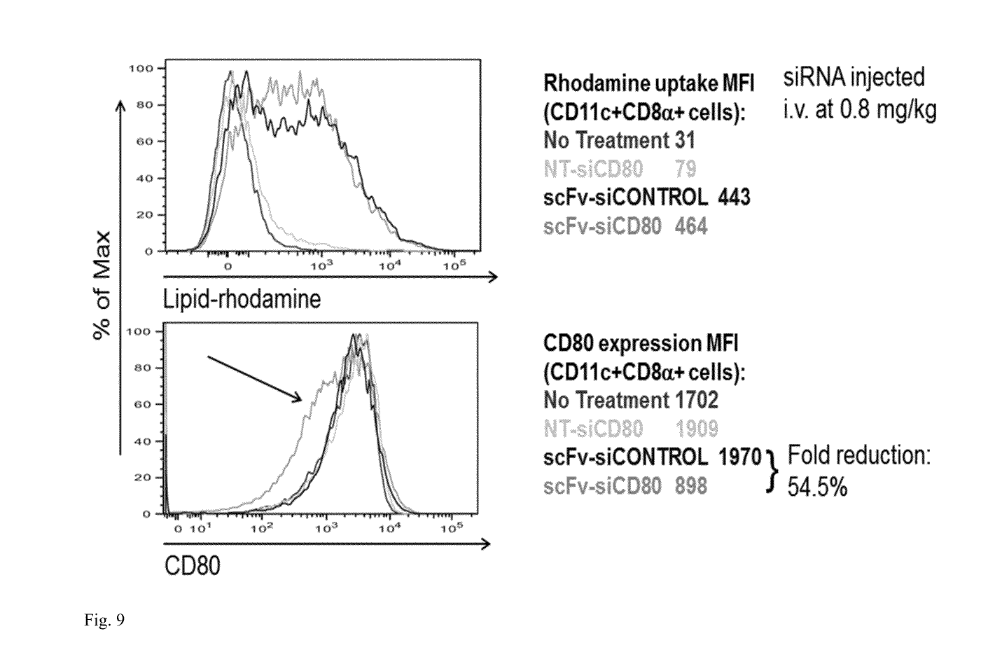

Having demonstrated specific uptake of scFv-LNPs by DCs in vivo, one goal was to show that this uptake was competent for specific gene silencing. LNPs (containing rhodamine B-labeled DOPE for tracking) were complexed with siRNAs specific for CD80 and these were injected into mice at 0.8 mg/kg. After 24 hrs LNP uptake and knockdown of CD80 was determined. As seen in FIG. 9, CD11c+DEC205+ cells took up similar levels of LNPs following injection with scFv-LNP containing either CD80− or control-siRNA (top panel, MFI: scFv-control=443 versus scFv-siCD80=464). Mice injected with non-targeted LNPs were taken up ˜5-fold less (MFI=79). Looking at levels of CD80 protein, it was found that mice given scFv-LNP encapsulated with CD80-specific siRNA showed ˜55% decrease in CD80 expression, when compared with scFv-LNP encapsulated with control-specific siRNA (FIG. 9, bottom panel. MFI: scFv-control=1970 versus scFv-siCD80=898).

### EXAMPLE 2

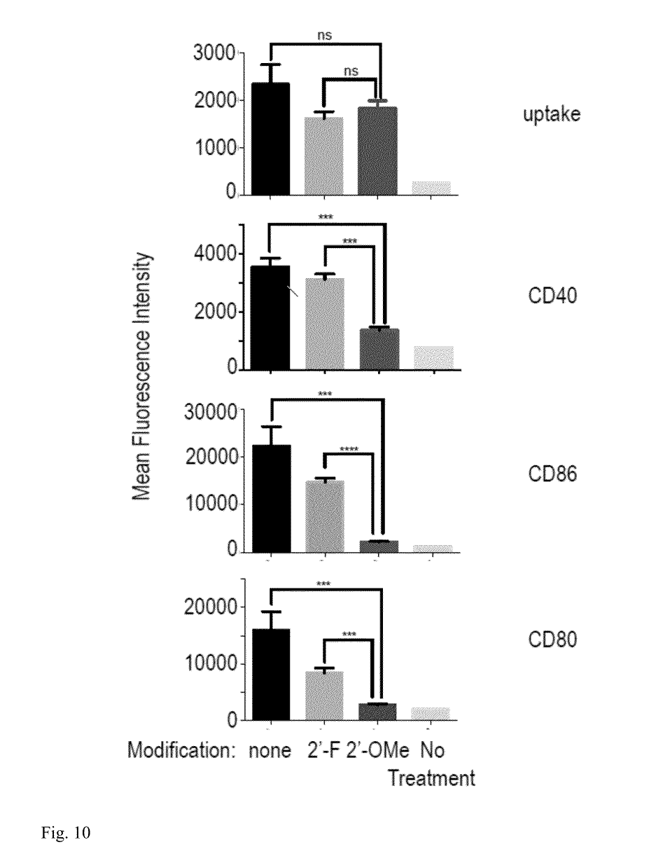

In further experiments, 2′-OMe modification of siRNAs was found to diminish dendritic cell activation following delivery via scFv DEC205-LNPs (see FIG. 10). Inclusion of siRNAs that are either unmodified or 2′-fluoro-modified stimulate activation of DC costimulatory molecules (CD80, CD86 and CD40). When the siRNA modification was changed to 2′-OMe, DC activation was no longer seen. Luciferase-specific siRNAs were used as a proof-of-principal to determine whether the 2′-OMe modification was sufficient to significantly decrease immune activation. This is an important consideration use of the targeted LNPs in autoimmune diseases.

### EXAMPLE 3

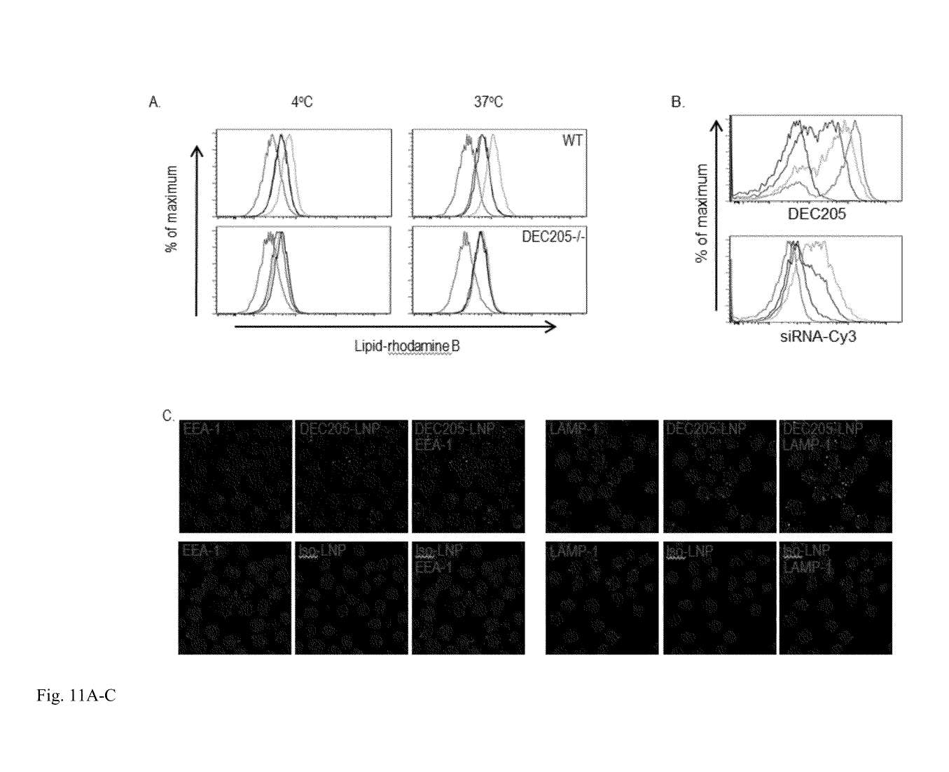

Confirming specificity and uptake pathway of DEC205-LNPs: Bone marrow-derived dendritic cells (BMDCs) derived from wild-type (WT) or DEC205-knockout (DEC205^{-/-}) mice were used to show that LNP uptake required the DEC205 receptor. As seen in FIG. 11A, at both 4° C. and 37° C. binding and uptake of LNPs coated with single chain antibody (scFv) specific for DEC205 occurred only in WT DCs, no binding or uptake was observed when DCs were derived from DEC205^{-/-} mice. LNPs coated with an isotype control antibody also did not bind WT DCs. Injection of WT and DEC205^{-/-} mice with scFv-DEC205 LNPs further confirmed the requirement for the DEC205 receptor for LNP binding and uptake (FIG. 11B). It was demonstrated that the uptake pathway used by DEC205-LNPs was similar to that reported for DEC205 antibody. The DEC205-LNPs were not observed at high levels in endosomes, but co-localized to lysosomes (FIG. 11C).

### EXAMPLE 4

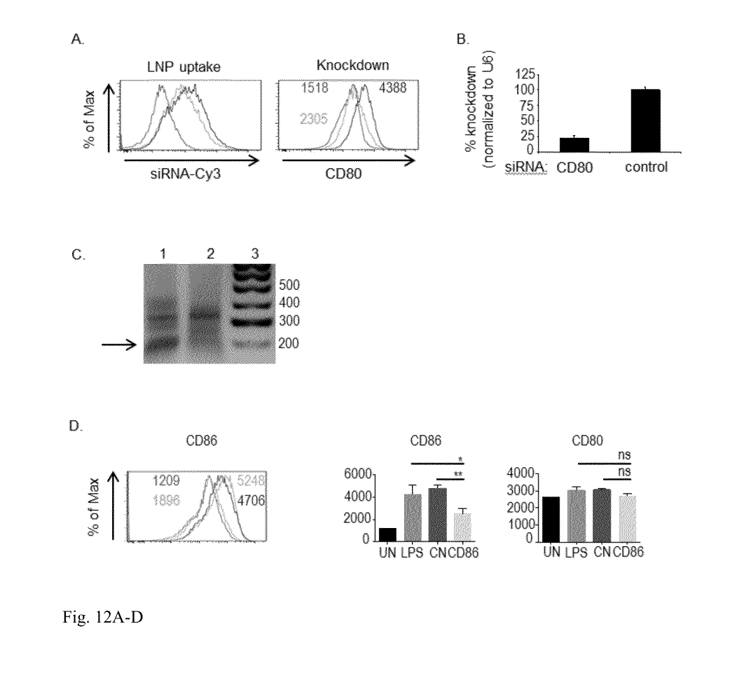

Expanding the specific genes that have been targeted to demonstrate generality of the approach: Initially it was shown that injection of DEC205-LNPs that contained siRNAs targeting CD80 resulted in uptake by DEC205+ DCs, leading to reduced expression of CD80 protein. It was further shown that following injection of DEC205-LNPs containing CD80-specific siRNAs, CD80 mRNA is reduced and that this mRNA knockdown occurs via an RNA interference (RNAi)-specific mechanism (FIG. 12A-C). Reduction in mRNA suggests that the observed knockdown occurs via RNAi. However, to demonstrate that the reduction in mRNA is RNAi-mediated, a PCR-based assay (5′ RACE) was used that is designed to amplify the cleavage product generated by the RNAi enzyme Argonaute (12). As seen in FIG. 12C, a band of the correct size is observed only in RNA extracted from DCs isolated from mice injected with DEC205-LNPs containing CD80-specific siRNAs.

As a use for these targeted LNPs is as a treatment for autoimmune disease, analysis was extended to another costimulatory molecule, CD86. As seen in FIG. 12D, reduced expression of CD86 protein was observed, with expression reduced to basal levels (left panel). The knockdown was specific: only CD86 expression was reduced (middle panel), expression of CD80 was unaffected (right panel). Quantitative PCR showed reduced expression of CD86 mRNA and we used 5′ RACE to demonstrate that mRNA cleavage is RNAi-mediated.

### EXAMPLE 5

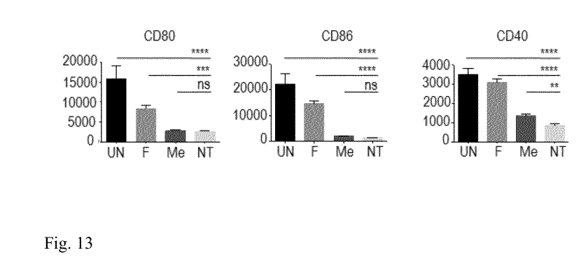

Using chemically-modified siRNAs to minimize unwanted immune responses: Injection of mice with DEC205-LNPs containing unmodified siRNAs resulted in DC activation. Therefore, siRNAs were encapsulated that contained either 2′-O-methyl (2′-Me) or 2′-fluorine (2′-F) substitutions on their backbone, into the DEC205-LNPs (13). FIG. 13 shows that when mice were injected with DEC205-LNPs containing unmodified siRNAs the DCs that took up the LNPs became activated, as demonstrated by expression of the costimulatory molecules CD80, CD86 and CD40. If siRNAs were modified with 2′-F some DC activation was observed, although it was generally less than that seen following injection of unmodified siRNAs. Injection of 2′-Me modified siRNAs showed minimal DC stimulation—only CD40 was slightly upregulated.

### EXAMPLE 6

Human- and murine-based experimental assays show different sensitivities following exposure to modified and unmodified siRNAs: As it was observed that delivery of unmodified siRNAs, and some modified siRNAs, to murine DCs in situ results in their activation, it was determined whether a simpler in vitro assay tconfimred whether an siRNA may cause non-specific immune activation. Although the ability of some siRNAs to elicit immune activation was abrogated following backbone modification (FIG. 13), this is not always the case.

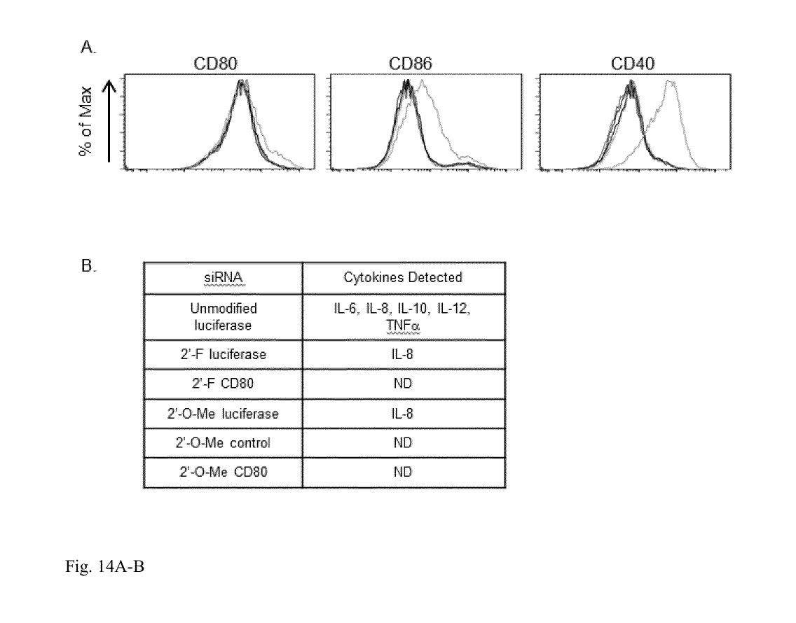

It was tested how a co-culture of LNP-encapsulated siRNAs with BMDCs affected DC activation. Both DEC-coated and uncoated LNPs were tested, which are taken up by cells similarly to liposomes used routinely as transfection reagents. No detectable upregulation of CD80, CD86 or CD40 was observed following culture with any of the LNP complexes tested (FIG. 14A). Therefore, we concluded that siRNA culture with BMDCs is likely not useful as a surrogate assay to determine the inherent ability of an siRNA to activate immune responses.

As the ultimate goal is to use LNPs as a delivery agent in humans, we switched to analyzing LNPs in human cell systems. Our scFv targets murine DEC205, therefore non-targeted (uncoated) LNPs were used in these assays. First, human peripheral blood mononuclear cells (PBMCs) were co-cultured with LNPs containing either unmodified, 2′-F or 2′-Me modified siRNAs. As seen in FIG. 14B only unmodified siRNAs elicited immune responses, and these were only observed when DCs were cultured with siRNAs at a concentration of 5 μM (unmodified luciferase). When the same siRNA sequence was either 2′-F—or 2′-Me-modified immune stimulation was minimal—only IL8 production was observed following culture with 5 μM siRNAs. Taking the assay one step further siRNA-encapsulating LNPs were cultured with human DCs. Unlike the results observed with human PBMCs, no DC activation was observed with either unmodified or modified siRNAs. These results suggest that i) splenic murine DCs are more sensitive to siRNA-mediated activation when compared with murine BMDCs, human PBMCs and DCs, ii) as LNPs were not coated they accessed a compartment in human PBMCs and DCs that did not contain immune-stimulating molecules and/or iii) the activation observed following PBMC culture is mediated by other immune cells.

### EXAMPLE 7

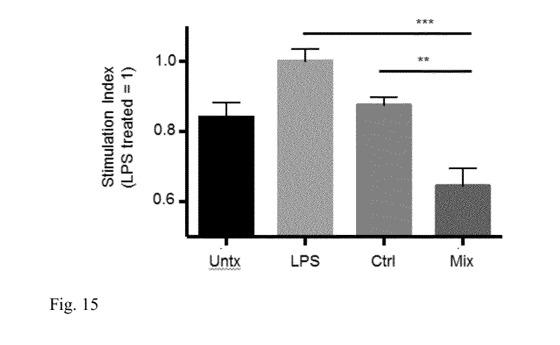

DEC205-LNPs containing siRNAs mediate functional gene-specific knockdown: FIG. 12 shows that DEC-LNPs containing siRNAs specific for the costimulatory molecules CD80 or CD86 can reduce protein expression close to basal levels. To determine whether this level of knockdown was sufficient to mediate reduced immune responses, a mixed lymphocyte response (MLR) assay was used to assess whether alloreactive responses were diminished following DEC205-LNP injection. C57L/6 mice were injected with DEC205-LNPs containing an equimolar mixture of siRNAs targeting the costimulatory molecules CD80, CD86 and CD40. One day later, DEC205+ DCs were isolated and co-cultured with T cells derived from Balb/c mice for a further 2 days. FIG. 15 shows that activation of alloreactive T cells was reduced by 36% when DCs were derived from mice injected with DEC205-LNPs containing CD80, CD86 and CD40 siRNAs, when compared with DCs derived from mice injected with LPS only (LPS versus Mix; P<0.001). Injection with non-targeted siRNAs did not result in a significant decrease in the alloreactive responses (LPS versus ctrl).

### REFERENCES

1. Davidson, B. L. & McCray, P. B., Jr. Current prospects for RNA interference-based therapies. Nat Rev Genet 12, 329-340 (2011).
2. Jayaraman, M., et al. Maximizing the potency of siRNA lipid nanoparticles for hepatic gene silencing in vivo. Angew Chem Int Ed Engl 51, 8529-8533 (2012).
3. Semple, S. C., et al. Rational design of cationic lipids for siRNA delivery. Nat Biotechnol 28, 172-176 (2010).
4. Banos, S. A. & Gollob, J. A. Safety profile of RNAi nanomedicines. Advanced drug delivery reviews (2012).
5. Zimmermann, T. S., et al. RNAi-mediated gene silencing in non-human primates. Nature 441, 111-114 (2006).
6. Frank-Kamenetsky, M., et al. Therapeutic RNAi targeting PCSK9 acutely lowers plasma cholesterol in rodents and LDL cholesterol in nonhuman primates. Proc Natl Acad Sci USA 105, 11915-11920 (2008).
7. Basha, G., et al. Influence of cationic lipid composition on gene silencing properties of lipid nanoparticle formulations of siRNA in antigen-presenting cells. Mol Ther 19, 2186-2200 (2011).
8. Novobrantseva, T. I., et al. Systemic RNAi-mediated Gene Silencing in Nonhuman Primate and Rodent Myeloid Cells. Molecular therapy. Nucleic acids 1, e4 (2012).
9. Inaba, K., et al. Tissue distribution of the DEC-205 protein that is detected by the monoclonal antibody NLDC-145. I. Expression on dendritic cells and other subsets of mouse leukocytes. Cell Immunol 163, 148-156 (1995).
10. Bonifaz, L., et al. Efficient targeting of protein antigen to the dendritic cell receptor DEC-205 in the steady state leads to antigen presentation on major histocompatibility complex class I products and peripheral CD8+ T cell tolerance. J Exp Med 196, 1627-1638 (2002).
11. Morrissey, D. V., et al. Potent and persistent in vivo anti-HBV activity of chemically modified siRNAs. Nat Biotechnol 23, 1002-1007 (2005).
12. Soutschek, J., et al. Therapeutic silencing of an endogenous gene by systemic administration of modified siRNAs. Nature 432, 173-178 (2004).
13. Choung, S., Kim, Y. J., Kim, S., Park, H. O. & Choi, Y. C. Chemical modification of siRNAs to improve serum stability without loss of efficacy. Biochem Biophys Res Commun 342, 919-927 (2006).

1. A composition comprising a lipid nanoparticle which comprises a lipid bi-layer, and a lipid having a single-chain variable fragment (scFv) attached thereto via a hydrophilic polymer wherein the scFv is directed against an antigen present on the surface of a cell.

2. The composition of claim 1, comprising an ionizable cationic lipid.

3. The composition of claim 1, comprising distearoylphosphatidylcholine (DSPC).

4. The composition of claim 2, wherein the ionizable cationic lipid comprises 1,2-DiLinoleyloxy-N,N-dimethylaminopropane (DLinDMA).

5. The composition of claim 1, comprising one or more of 1,2-dilinoleyl-4-(2-dimethylaminoethyl)[1,3]-dioxolane (DLinKC2-DMA), dilinoleyl-methyl-4-dimethylaminobutyrate (DLin-MC3-DMA), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl phosphatidyl ethanolamine, 16-O-dimethyl phosphatidyl ethanolamine, 18-1-trans phosphatidyl ethanolamine, 1-stearoyl-2-oleoyl-phosphatidyethanolamine (SOPE), N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), and N,N-dimethyl-2,3-dioleyloxy)propylamine (DODMA), and 1,2-Dilinolenyloxy-N,N-dimethylaminopropane (DLendMA).

6-8. (canceled)

9. The composition of claim 1, wherein (i) a nucleic acid molecule, (ii) a small organic molecule, (iii) vaccine or (iv) antigen is present in the lipid nanoparticle.

10. (canceled)

11. The composition of claim 9, wherein the nucleic acid molecule is present and is a siRNA, shRNA, plasmid, mRNA, miRNA or ncRNA.

12. The composition of claim 1, wherein the scFv is an anti-DEC-205 scFv, anti-Cd11c scFv, anti-33d1 scFv or anti-Clec9A (DNGR1) scFv.

13-26. (canceled)

27. A method of delivering a small molecule, a nucleic acid, a vaccine or an antigen to a cell expressing a cell-surface antigen, comprising contacting the cell with the composition of claim 1, wherein the cell surface antigen is the antigen to which the scFv is directed, in an amount effective to deliver a small molecule, a nucleic acid, a vaccine or an antigen to a cell expressing a cell-surface antigen.

28. A method of increasing the efficacy of a predetermined dose of a RNAi nucleic acid administered to a subject comprising delivering the RNAi nucleic acid to a cell expressing a cell-surface antigen, comprising contacting the cell with the composition of any of claim 1, wherein the cell surface antigen is the antigen to which the scFv is directed, in an amount effective to deliver a RNAi nucleic acid to a cell expressing a cell-surface antigen and thereby increase the efficacy of the predetermined dose of a RNAi nucleic acid.

29. The method of claim 27, wherein the cell is a dendritic cell.

30. The method of claim 27, wherein the nucleic acid is delivered.

31. (canceled)

32. The method of claim 30, wherein the nucleic acid is a siRNA, shRNA, plasmid, mRNA, miRNA or ncRNA.

33. The method of claim 32, wherein the nucleic acid is an immune-modulatory siRNA.

34. A method of treating an immune system disorder in a subject comprising administering to the subject an amount of the composition of claim 1, wherein the siRNA is an immunomodulatory siRNA or the small organic molecule is an immunomodulatory small organic molecule.

35. The method of claim 34, wherein the scFv is an anti-DEC-205 scFv.

36. The method of claim 34, wherein the siRNA is delivered.

37. The method of claim 34, wherein the immune system disorder is an autoimmune disorder.

38. The method of claim 24 any of claims 34-37, wherein the siRNA is directed against a human CD86, a human CD40 or a human CD80.

39. The method of claim 34, wherein the subject is a human.
